(19) United States
(12) Patent Application Publication
(10) Pub. No.: US 2002/0034739 A1
(43) Pub. Date: Mar. 21, 2002

(54) PEPTIDES CHARACTERISTIC OF CERTAIN TUMORS

(76) Inventors: ROBERT I. LEHRER, LOS ANGELES, CA (US); CHENGQUAN ZHAO, LOS ANGELES, CA (US); BENJAMIN J. GLASGOW, LOS ANGELES, CA (US)

Correspondence Address: KATE H. MURASHIGE, MORRISON & FOERSTER LLP, 3811 VALLEY CENTRE DRIVE, SUITE 500, SAN DIEGO, CA, 92130-2332, US

(21) Appl. No.: 09/110,716

(22) Filed: Jul. 7, 1998

(51) Int. Cl. C12Q 1/68

(52) U.S. Cl. 435/6

(57) ABSTRACT

Peptides that are characteristic of tumors of the steroid regulated organs are disclosed. The peptides are useful for the production of antibodies that may in turn be used to detect metastases of these tumors. The antibodies may also be used for purifying the peptides.

The nucleotide sequences encoding these peptides are also useful for the design of oligonucleotide probes and primers for the detection of expression of the genes encoding these peptides.

### TECHNICAL FIELD

The invention relates to peptides that are present in tumors and which, when cells are shed by solid tumors, can be used to detect metastases. More specifically, the invention concerns lipophilins A, B and C-peptides which appear to be related to prostatein and to means for their detection.

### BACKGROUND ART

An extended family of peptides that appears to be associated with hormonally regulated organs has been disclosed piecemeal over the last two decades. Members of this family include, most prominently, prostatein, which is a tetrameric rat protein containing three different peptides designated C1, C2 and C3. The tetramer has two noncovalently associated heterodimers with the composition (C1 :C3)/(C3:C2); each of the heterodimers is stabilized by three intermolecular cystine residues. Prostatein is the most abundantly secreted protein of the rat ventral prostate gland, and accounts for 40% of its total protein secretion. It has alternatively been called “prostatic binding protein,” “prostatic α-protein,” “estramustine binding protein,” “prostatic secretion protein,” and “prostatic steroid binding protein.” The synthesis of prostatein in the rat prostate is dramatically controlled by androgens and no human prostatic analog has been found. However, because prostatein is so highly regulated by androgens, it has been suggested that prostatin C3-mRNA is a useful marker for in vitro studies of androgen agonists and antagonists. Shao, T. C. et al. Prostate (1990) 17:41-55.

Each of the genes of C1, C2 and C3 has been cloned and sequenced: Viskochil, D. H. et al. J Biol Chem (1983) 258:8861-8866; Parker, M. et al. Ann NY Acad Sci (1984) 438:115-124; Hurst, H. C. et al. EMBO J (1983) 2:769-774.

Other proteins in this family are the uteroglobins which are also called “CC 10” or “Clara cell proteins.” The first uteroglobin was found as a small secretory protein of rabbits synthesized mainly by uterine glandular and lumenal epithelial cells as well as by Clara cells of the lung. Rabbit uteroglobin and human Clara cell protein are closely related since their cDNAs show 75% overall identity in the coding regions. The 70-residue uteroglobin monomers form antiparallel homodimers stabilized by disulfide bonds between cys3-cys69′ and cys69-cys3′. Expression of uteroglobin is differentially regulated by steroid hormones—progesterone in the endometrium, testosterone in the epididymis and cortisol in the lung. Rabbit uteroglobin homodimers can also bind calcium ions and bind progesterone with high affinity under reducing conditions. They are potent inhibitors of phospholipase A2.

The counterpart human CC10 protein is produced by both pulmonary cells and uterine epithelial cells; the human CC10 gene has been localized to chromosome 11q11-qter. Mature CC10 protein is a 16 kD homodimer whose 70 residue monomers are composed of four helixes joined by two antiparallel disulfide bonds. The CC10 dimer encloses a large internal cavity that can bind hydrophobic molecules, including phosphatidyl choline and phosphatidyl inositol; CC10 is a potent inhibitor of phospholipase A2 also. The active human counterpart to rabbit uteroglobin was isolated from human blood by Aoki, A. et al. Mol Hum Reprod (1996) 2:489-497. See also Zhang, Z. et al. DNA and Cell Biol (1997) 16:73-83. The corresponding protein (uteroglobin/CC10) was also isolated from monkey lung lavage by Hashimoto, S. et al.

Am J Res Cell Mol Biol (1996) 15:361-366. The availability of recombinant human uteroglobins has permitted studies on its biological role. See Kundu, G. C. et al. Proc Natl Acad Sci USA (1996) 93:2915-2919; Leyton, J. et al. Cancer Res (1994) 54:3696-3699. The nature of progesterone binding to this protein has also been studied by Dunkel, R. et al. Protein Engineer (1995) Jan 8(1):71-79.

A review of the uteroglobin family is authored by Miele, L. et al. J Endocrinol Invest (1994) 17:679-672. Uteroglobin is also the subject of U.S. Pat. No. 5,696,092 directed to inhibiting metastasis of epithelial cell-derived cancers. Antiinflammatory peptide analogs based on the uteroglobin structure are disclosed in U.S. Pat. No. 5,480,869.

An additional member of the family is human mammaglobin which is described in U.S. Pat. No. 5,668,267 as a specific breast cancer protein.

Other members of this family include the protein encoded by FHG22 isolated from a female-male subtracted cDNA library of a hamster, major cat allergen FELdI (chain 1) and mouse cell androgen binding proteins. Unidentified proteins that appear on two-dimensional electrophoresis of human ejaculates may be related to the proteins herein. Rui, H. et al. Int J Androl (1984)7:509-520. Similarly, several proteins of unknown structure have been noted in two-dimensional electrophoresis of human prostatic fluid (Carter, D. B. et al. Prostate (1982) 3:27-33).

Subsequent to the work described herein, a paper by Molloy, M. P. et al. Electrophoresis (1997) 18:2811-2815 (December issue) described mapping the protein components of normal human reflex tears. One of the proteins isolated on a two-dimensional gel was named lachryglobin. Lachryglobin was subjected to a amino acid analysis and Edman sequencing; the N-terminal 68 amino acid sequence of lachryglobin is identical to the N-terminal amino acid sequence of lipophilin C disclosed herein. Molloy et al. suggest that as lachryglobin displays strong homology with mammaglobin which is overexpressed in breast cancer, the discovery of this homolog in tears offers the potential for disease diagnosis by screening tear fluid proteins.

### DISCLOSURE OF THE INVENTION

The invention provides materials and methods for prognosis and diagnosis of carcinomas associated with hormonally regulated tissues such as cancers of the prostate, testis, uterus, breast, and ovary. Cells of these organs express the genes for one or more of the lipophilin peptides described herein, while it is clear that genes encoding these peptides are not expressed by the bone marrow or by blood cells. Thus, the presence of the lipophilins or the associated mRNA in the bloodstream is evidence for expression of the genes encoding these peptides and is indicative of blood-borne dissemination of carcinoma cells originating from these organs. This information could prove decisive in electing to offer adjuvant chemotherapy to patients who might otherwise not receive it. The availability of the peptides of the human lipophilin family and of the nature of the nucleotide sequence natively encoding them provides the basis for the design of tools for the detection of metastasized cells in the bloodstream, and biopsied tissues, e.g., bone marrow and other organs. Determining the presence or absence and amount of metastasized cells offers an opportunity for prognosis of the progression of the disease in a subject and also permits a mechanism for monitoring therapeutic protocols. A diminution in metastases when seen subsequent to administration of such protocols indicates probable success. Failure of a protocol to diminish the level of metastasis indicates that alternatives may be more successful.

Accordingly, in one aspect, the invention is directed to peptides having the amino acid sequence of human lipophilin A, B and C and their allelic variants, as well as to fragments of these peptides which contain at least one immunogenic epitope of these peptides. These peptides are useful to generate antibodies which can then be used for purification and detection of these peptides in suitable test samples.

In another aspect, the invention is directed to nucleotide sequences encoding these peptides and means for their recombinant production.

In still another aspect, the invention is directed to oligonucleotide probes and primers that are useful in detecting the transcription products of the genes encoding the human lipophilin family. These oligonucleotides are useful in the detection of the expression products of the lipophilin genes and can, like the antibodies described above, be employed for detection of metastases in body fluids and biopsied tissues.

### BRIEF DESCRIPTION OF THE DRAWINGS

FIG. 1 shows an HPLC trace indicating the position of the lipophilin A-C dimer purified from human tears.

FIG. 2 shows the amino acid sequences of lipophilins A and C as determined from the tear isolate.

FIG. 3 shows primer sequences for cDNA cloning, PCR and amplification of the lipophilins as well as of mammaglobin and human uteroglobin.

FIG. 4 shows the nucleotide sequence of the cDNA encoding lipophilin A and the deduced amino acid sequence.

FIG. 5 shows the nucleotide sequence of the cDNA encoding lipophilin B and the deduced amino acid sequence.

FIG. 6 shows the nucleotide sequence of the cDNA encoding lipophilin C and the deduced amino acid sequence.

FIG. 7 shows the alignment of the nucleotide sequences for cDNA encoding lipophilins A and B with that of rat prostatein C2.

FIG. 8 shows the alignment of the cDNA nucleotide sequence encoding lipophilin C with those of mammaglobin and rat prostatein C3.

FIG. 9 shows the alignment of the primary amino acid sequences of lipophilins A and B with rat prostatein C 1 and C2 and of lipophilin C with rat prostatein C3 and mammaglobin.

FIG. 10 is a dendrogram showing the relationship of the nucleotide sequences encoding the propeptides of lipophilins A-C and related peptides.

FIGS. 11A and 11B show the expression of lipophilins A, B and C as well as of mammaglobin, uteroglobin and β-actin in various tissues as determined by reverse transcription PCR.

FIG. 12 shows the expression of lipophilins B and C in various tumor cell lines as determined by reverse-transcription PCR.

### MODES OF CARRYING OUT THE INVENTION

The tools provided to carry out prognosis and diagnosis of malignant tumors associated with hormonally regulated organs are based on the surprising discovery in human tears of peptides that are related to peptides secreted by the rat prostate gland. It has been found, also unexpectedly, that the genes encoding these peptides are expressed preferentially in hormonally regulated and hormonally responsive organs, but not by the bone marrow or blood cells where expression would interfere with their use in detecting metastases of tumors and in assessing the effectiveness of chemotherapeutic or other protocols designed to effect regression of a primary tumor. It will be apparent, therefore, that if the bloodstream or bone marrow contain evidence of expression of the genes encoding these peptides, cells in which such expression is occurring must be present. Thus, metastatic manifestations of the original tumor can be detected and used to assess the progress of the disease and to monitor the progress of treatments administered to the subject.

The peptides of the invention, human lipophilins A, B and C are closely related peptides encoded by closely related nucleotide sequences. A heterodimer of lipophilins A and C present in human tears provided the information that resulted in the recovery of the cDNA encoding these peptides from a lachrymal cDNA library and, unexpectedly, the recovery of a cDNA encoding lipophilin B from a human uterus cDNA library. The availability of these peptides and nucleotide sequences provides useful tools for detecting the presence of metastatic tissue in the bloodstream of human subjects, as further described below.

Metastases of carcinomas of hormonally responsive and/or hormonally regulated tissue such as uterus, ovary, prostate, testis, breast, kidney and thymus can be determined by virtue of the expression by cells of these tumors of the gene encoding one or more of the human lipophilins against a background of hematopoietic cells that does not result in the expression of these genes. The kidney and thymus are responsive to steroid hormones—the kidney to aldosterone and the thymus to corticosteroids.

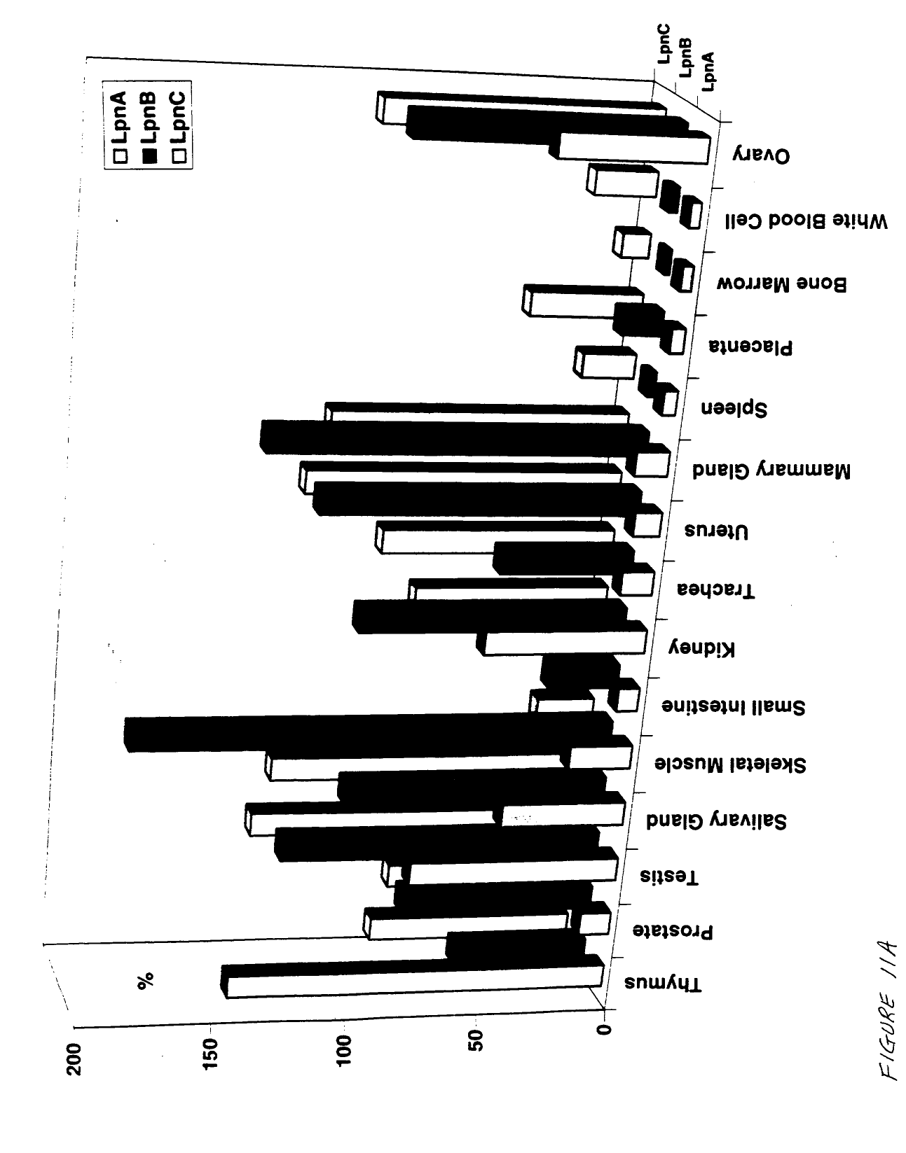

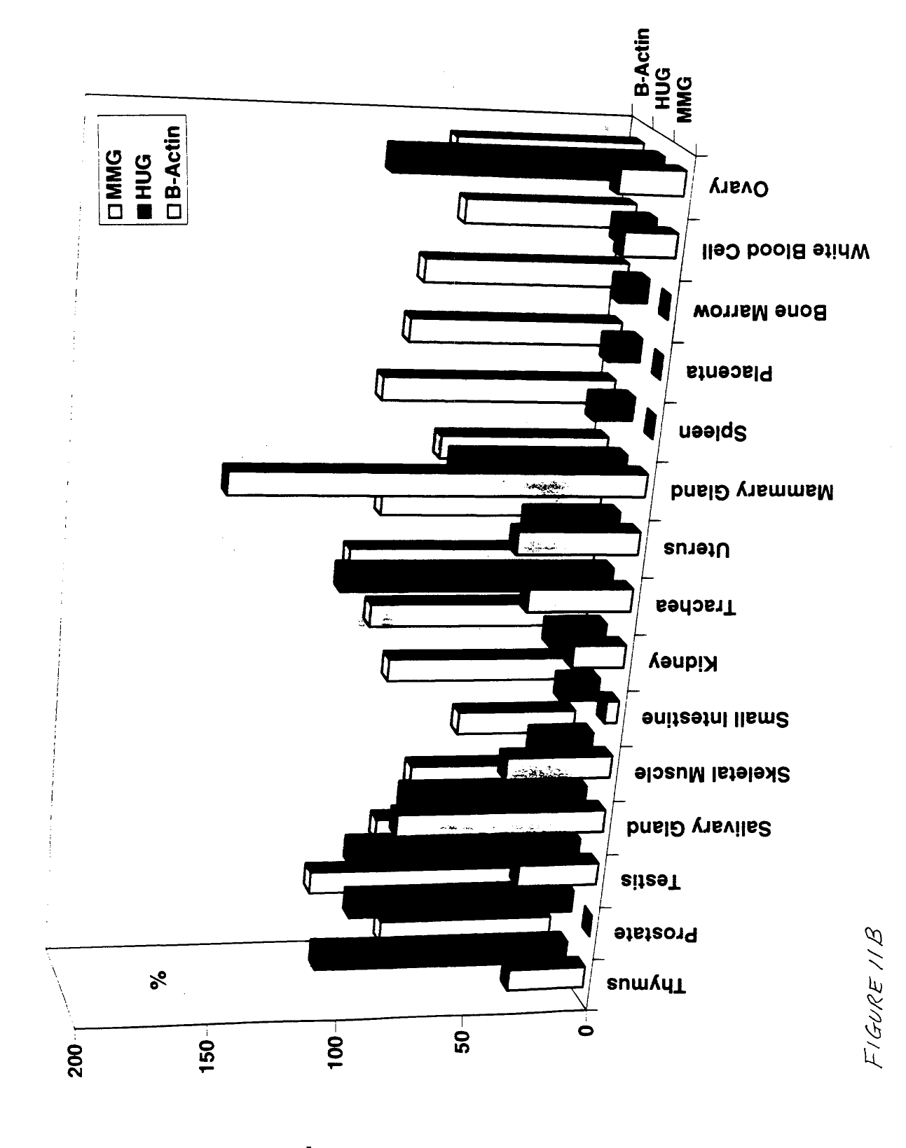

FIGS. 11A and 11B show the pattern of expression lipophilins A, B and C genes in various human tissues. The representation in FIGS. 11A and 11B shows the results of RT-PCR which was quantitated by densitometric analysis and normalized to the expression of β-actin in the same tissue. It is apparent from FIG. 11A that spleen, bone marrow, and white blood cells do not express substantially any of the lipophilin genes while significant expression of the lipophilin B gene is exhibited by skeletal muscle and salivary glands. These tissues, however, do not represent troublesome background in the detection of metastases in blood.

The presence of metastases is measured by the presence of cells expressing lipophilin genes in the bloodstream, bone marrow or other body fluid. Any method that results in the detection of cells that express lipophilin A, B or C alone or in combination in blood or a fraction of blood used in the analysis, is within the scope of the invention. A number of methods are currently known for detection of expression; additional methods may be devised in the future.

One group of methods relies on the detection of the protein itself. Metastasized cells that are shed into the bloodstream or other body fluid or bone marrow may secrete lipophilin A, B or C or may contain the peptide intracellularly. If the protein is intracellular, prior extraction to lyse the cells may be required before applying the appropriate analytical technique. Methods to prepare samples of blood, serum, plasma or other body fluids so as to make the peptides present in such fluids available for assay are well known in the art. Any suitable fraction of the fluid may be used. Preliminary separation steps to isolate fractions which are enriched in the desired peptides, if present, may be performed if desired.

The simplest known method to detect the presence of a peptide in a body fluid rests on an immunoassay approach. Various protocols for immunoassays are known, and any suitable protocol can be employed. Typically, such assays depend either on detection directly of a complex formed between the peptides or protein to be detected and an antibody or a fragment of an antibody immunoreactive with it or depend on competition between a peptide in the fluid to be analyzed with a labeled competitor for the antibody or fragment. Various labels for detection of the complex can be used, including enzyme labels, fluorescent labels, chromogenic labels, radioisotopes, or any other property that can be measured quantitatively or qualitatively. The typical assay is conducted on a solid phase as a heterogeneous assay, but homogeneous assays for immunocomplex formation are also known.

Antibodies can be prepared to the peptides of the invention by a variety of known techniques. Typically, the peptide or an immunogenic fragment thereof containing at least one epitope is administered to a subject capable of forming an immune response such as a rat, rabbit, guinea pig, mouse, or sheep. It may be desirable to couple the peptide or fragment to an additional carrier to enhance its immunogenicity. Techniques for preparing such conjugates are well known and often involve chemical linkers such as those manufactured by the Pierce Chemical Company, Rockford, Ill. Methods and protocols for enhancing immunogenicity and eliciting an antibody response are well known. When suitable antibody titers are obtained, polyclonal compositions which are monospecific for the peptides can be obtained using the peptides as affinity ligands. Alternatively, monoclonal antibody preparations specific for the peptides are prepared using standard hybridoma technology. Spleen cells or peripheral blood lymphocytes are harvested from the immunized mammal and immortalized using fusion with a myeloma or other methods such as infection with EBV.

The immortalized cells are then screened for production of the appropriate monoclonal antibodies by immunoreaction with the corresponding peptide. The monoclonal antibodies may be produced by these immortalized cells and recovered for use in the methods of the invention or may be produced recombinantly by recovering the genes and producing the antibodies in bacteria, yeast or other suitable recombinant host cells, such as insect cells. The availability of recombinant techniques permits the production of modified forms of the antibodies, such as Fv single-chain antibodies as well as immunologically reactive fragments of the antibodies such as the F_{ab}, F_{ab′}, or F_{(ab′)}_{₂} fragments. These fragments can also be prepared directly from the antibodies themselves by suitable proteolytic techniques. Thus, by “immunoreactive fragments” of antibodies is meant those portions of the variable regions which confer antigen specificity, whether prepared by proteolysis of the antibodies or by recombinant techniques.

For use in the immunoassays of the invention, either whole antibodies may be used, fragments which immunoreact with the target may be used, or genetically engineered single-chain forms such as Fᵥ forms may be used. As used herein, “immunologically reactive portion thereof” refers to such fragments or genetically engineered versions which retain the ability to recognize the target antigen.

These antibodies or fragments are also useful in immunohistochemical techniques performed on tissue samples or bone marrow. This technique permits identification of bone metastatic cells or small metastatic foci at these locations.

In addition to the use of antibodies for immunoassays, the peptides produced by metastatic cells can be detected by a variety of other, perhaps less convenient, means such as assessing the fluids or extracts using separation techniques such as RPLC, two-dimensional thin-layer chromatography, SDS-PAGE, agarose electrophoresis and the like. A variety of chromatographic techniques can be used to isolate and identify the peptides produced.

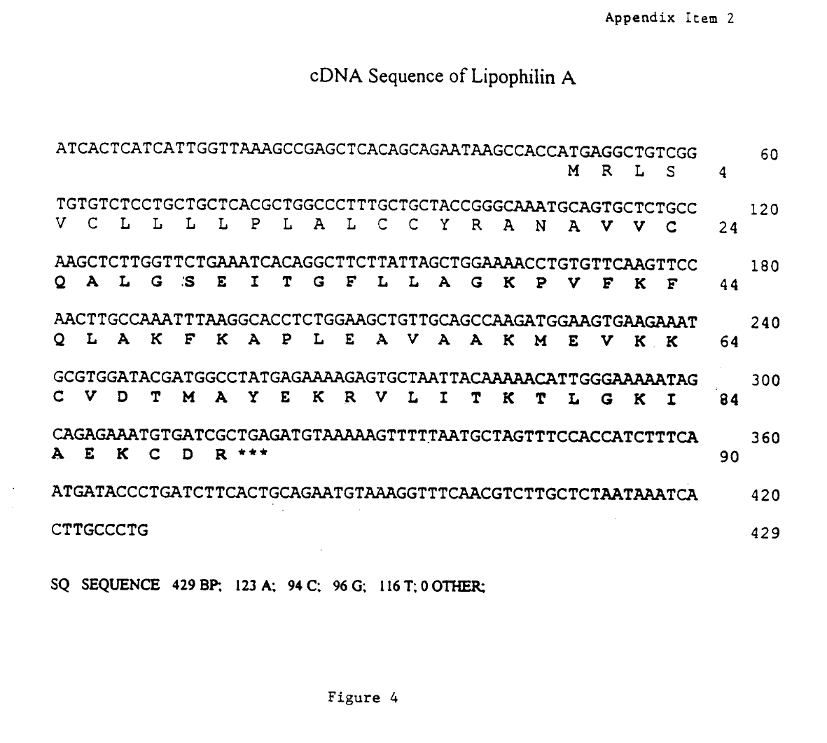

In a second approach, the mRNA expression product of the genes encoding the lipophilin peptides is detected. The mRNA can be detected directly by hybridization to probe or by PCR amplification, or more typically the mRNA can be reverse-transcribed into cDNA and this, in turn, detected by these means. By “mRNA corresponding to lipophilin A, B or C or reverse transcript” is meant the complete mRNA transcribed from the gene encoding these peptides, including upstream and downstream untranslated portions thereof. The nucleotide sequences of the mRNA/cDNA corresponding to the human lipophilins A, B and C are shown in FIGS. 4-6 herein. Allelic variants of these nucleotide sequence are also contemplated.

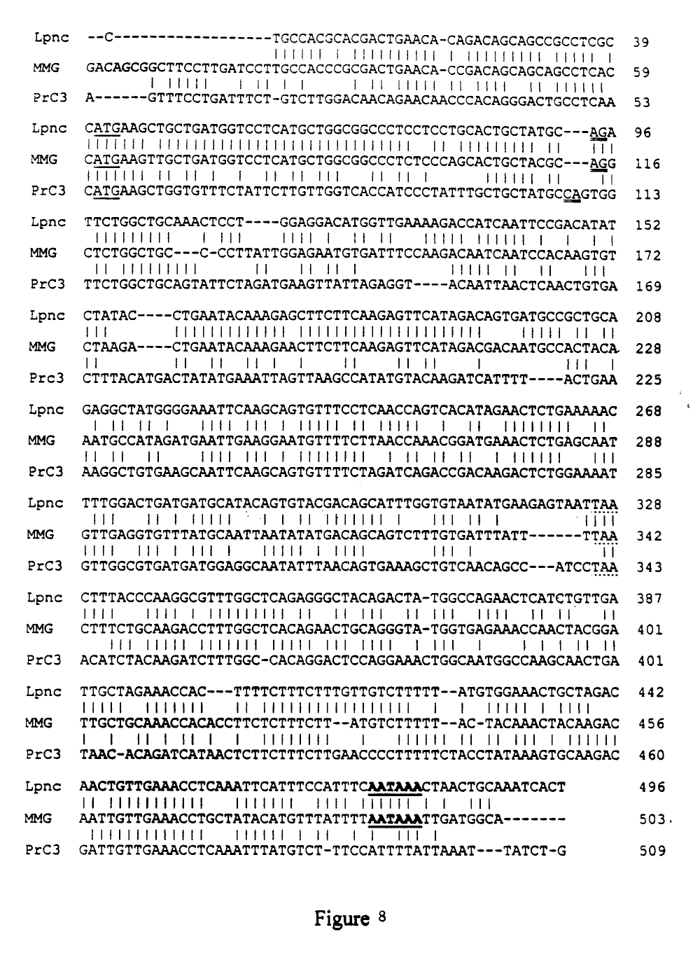

The mRNA can be optionally purified and subjected to agarose electrophoresis or other separation techniques and then detected by hybridization to suitable probe. The conditions for hybridization will depend on the length of the probe and the nature of the possible mRNA contaminants. Since the nucleotide sequences encoding the human lipophilins are disclosed herein, completely homogeneous probes can readily be devised and regions of known homology to other related proteins such as mammaglobins and uteroglobins can be avoided. Accordingly, the stringency of the hybridization techniques is adjusted to discriminate between the level of homology to the target sequence as compared to possible competing sequences. Referring, for example, to FIG. 8, regions of homology of the nucleotide sequence encoding lipophilin C to mammaglobin can be determined and avoided. Suitable probes are typically 9-20 nucleotides in length.

Alternatively, the mRNA can be detected as an amplification product using suitable primers. Again, primers are chosen to avoid duplication to possible competing nucleotide sequences. The primers and probes are chosen according to the subject nucleic acid to be detected—whether the mRNA per se or cDNA transcribed from it.

Means of preparing suitable samples from blood or other body fluids which will contain the desired peptide or nucleic acid are well known in the art. The peptide is provided in a form suitable for complexing to antibody and mRNA in a form suitable for amplification or hybridization to probe.

The peptides of the invention, needed for the preparation of antibodies can readily be prepared using standard solid-phase or solution-phase peptide synthesis techniques which are now commercially available. In addition, these peptides may be prepared recombinantly using appropriate encoding nucleotide sequences which can, in turn, be prepared using standard oligonucleotide synthesis methods. Methods of preparation for the components of the invention—the peptides, nucleic acids, and antibodies are well understood in the art once the amino acid sequences or nucleotide sequences, respectively, are disclosed.

The discussion above focuses mainly on body fluids where the general methodologies described readily apply. Assessment can also be done on histochemical samples of biopsied tissues such as salivary glands, skeletal muscle and thymus. These tissues also express lipophilin genes and methods which involve homogenization, detergent extraction, and the like would not be workable.

However, these biopsied tissues can be assessed histochemically without disruption and tumor cells can be detected using immunohistochemistry or by hybridization to antisense RNA. Metastases to such organs are, however, relatively rare. Nevertheless, detection of metastases to biopsied tissues as well as in the context of body fluids is included with the scope of the invention. The combination of biopsied tissues and body fluids includes, therefore, blood, spinal fluid, bone marrow, various organs, and the like.

As shown in the Examples below, the lipophilin B gene has been localized to chromosome 10, band 22-23. Chromosome 10q22-23 harbors an antioncogene implicated in prostate cancer (Komiya, A. et al. Genes, Chromosomes and Cancer (1996) 17:245-253) and has also been implicated in Cowden's Syndrome, an autosomal dominant hamartoma syndrome associated with its high risk of thyroid and breast cancer (Dahia, P. L. et al. Cancer Res (1997) 57:4710-4713; Nelan, M. R. et al. Nature Genetics (1996) 13:114-116). Lipophilin B may therefore represent the product of a tumor suppressor gene adjacent to the PTEN/MMA-C 1 locus on 10q23 described by Singh, B. et al. Genes, Chromosomes and Cancer (1998) 21:166-177; Nihei, N. et al. Genes, Chromosomes and Cancer (1995) 14:112-119; Whang, Y. E. et al. Proc Natl Acad Sci USA (1998) 95:5246-5250).

The possibility that both lipophilin B and lipophilin C represent tumor suppressors is substantiated by the data set forth hereinbelow in Example 8 which demonstrate that these proteins are produced in prostatic cancer cell lines at levels lower than those characterizing normal prostate. The known tumor suppressor PTEN/MMAC1 was shown to be inactivated in advanced human prostate cancer through loss of its expression. Whang, Y. E. et al., Proc Natl Acad Sci USA (supra). This report showed that only one of ten prostate tumors examined contained a homozygous deletion of PTEN/MMAC 1 and that no mutations were found in the coding region of the remaining 9 xenografts. However, 5 of the 10 xenografts showed reduced or absent PTEN/MMAC 1 expression. In the present case, as shown hereinbelow, prostate cancer cell lines and xenografts also show reduced expression of lipophilin B and lipophilin C. Such abnormal expression would be expected of a tumor suppressor.

The following examples are intended to illustrate but not to limit the invention.

### EXAMPLE 1

### Isolation of Lipophilin A-C from Tears

Healthy adult volunteers were briefly exposed to the vapors of freshly minced onions. Over the next 5-10 minutes, 100-250 μl of stimulated tears was collected and stored at −20° C. until used.

Tears were pooled and subjected to RP-HPLC on a 10×250 mm, Vydac 218TP510 C-18 column (Vydac, the Separations Group, Hesperia, Calif. ), using a linear gradient of acetonitrile (CAN) in 0.1% trifluoroacetic acid (TFA), that increased in CAN concentration by 1%/min. Fractions were subjected to SDS-PAGE on gels with 16.5% acrylamide with and without reduction by dithiothreitol (DTT). The previously unidentified peptides were assessed by preliminary N-terminal sequencing of isolated peptides transferred to PVDF membranes. One such transferred peptide yielded two residues in each of the first 25 cycles, whereas cycles 26-42 provided a single sequence: AKFKATLXAVAAKMEVK. A composite query sequence containing more abundant residue in each of the first 25 cycles followed by the unique sequence from cycles 26-42 was constructed and used as the basis for a BLAST search. No human homolog was found; but 37.5% of residues were identical to the C1 component of rat prostatein.

Fractions containing this previously unidentified protein were further purified by RP-HPLC on a 4.6×250 mm Vydac 218TP54 C-18 column, using various linear gradients of acetonitrile in 0.1% TFA to obtain lipophilin A-C dimers.

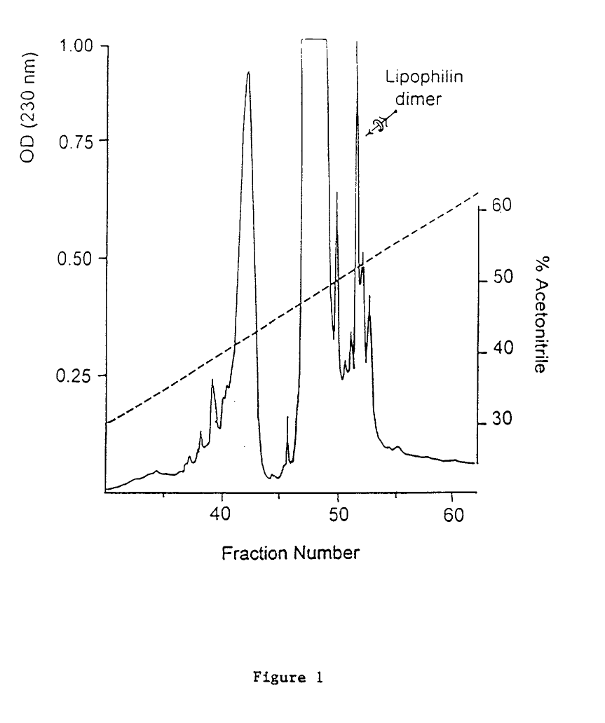

The lipophilin A-C peak that emerged from the purification column was detected by SDS-PAGE with and without DTT reduction corresponding to the previously sequenced peptide. The elution pattern resulting in the A-C dimer is shown in FIG. 1.

The dimer was then further purified to apparent homogeneity on a small C18 column using 0.13% heptafluorobutyric acid (HFBA) as the ion-pairing agent. The mass of the purified dimers was 16,424.01±1.17 by ESI-MS. Approximately 414 μg of dimer was obtained from 9 ml of pooled human tears; its concentration is thus in excess of 45 μg/ml.

The HPLC-purified lipophilin A-C (50 μl, 1 mg/ml) was resuspended in a buffer containing 6M guanidine.HCl, 20 mM EDTA and 0.5 M Tris, pH 8.07. After flushing with nitrogen and incubation in a 52° C. water bath for 10 minutes, freshly prepared DTT was added in approximately 2,000-fold molar excess of lipophilin. The samples were again flushed with nitrogen, sealed and incubated for 2.5 hours at 52° C. Then, additional DTT was added, followed in 45 minutes by the addition of glacial acetic acid (5% final concentration) to stabilize the reduced products. These were resolved by RP-HPLC on a 4.6±250 mm C18 column, with a linear CAN gradient in 0.1% TFA.

Alternatively, after reduction, the mixture was cooled to room temperature, protected from light, and iodoacetamide was added in a three-fold molar excess, relative to DTT. After 10 minutes, DTT (equal in amount to the first addition) was added to quench unreacted iodoacetamide. The carboxamidomethylated (CAM) derivatives of lipophilins A and C were then separated and purified by RP-HPLC.

By comparing the mass of lipophilins A and C with and without CAM modification, it was determined that both lipophilin A and lipophilin C contained three cysteine residues. The measured mass of the parent lipophilin heterodimer (16,424.01) corresponded to the calculated mass of a heterodimer that contained one molecule of component A (7,574.69) linked to one molecule of component C (8,854.94).

### EXAMPLE 2

### Determination of Amino Acid Sequence

The separated heterodimer and components, including CAM derivatives were examined for structure as follows:

Lipophilin heterodimer and purified components A and C were tested for glycosylation with the ECL glycoprotein detection kit (Amersham, Arlington Heights, Ill.), using lactoferrin standards. Neither lipophilin component was glycosylated.

N-terminal sequencing was performed with a Porton Model 2090E sequencer (Beckman Instruments, Fullerton, Calif.), either directly, or after transferring the CAM-modified peptide to a polyvinylidene fluoride (PVDF) membrane (Millipore, Bedford, Mass.). In addition, CAM-modified component A or C was dissolved at 0.5 mg/ml in 0.1 N HCl and cyanogen bromide was added in slight excess, by mass, to peptide. The mixture was incubated with occasional shaking for 30 hours at room temperature before performing RP-HPLC to obtain peptide fragments for mass determinations or sequencing.

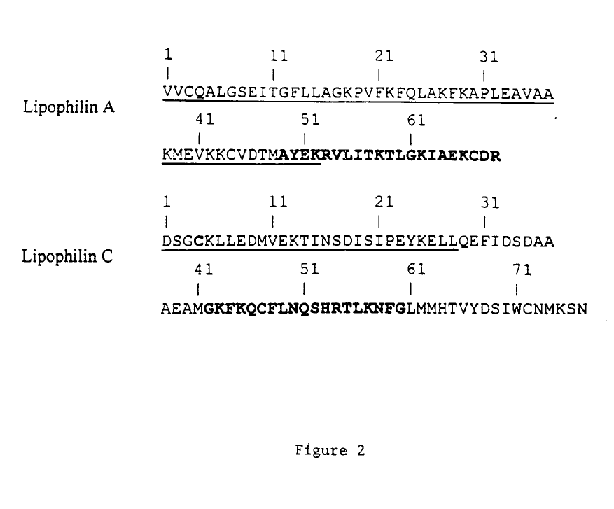

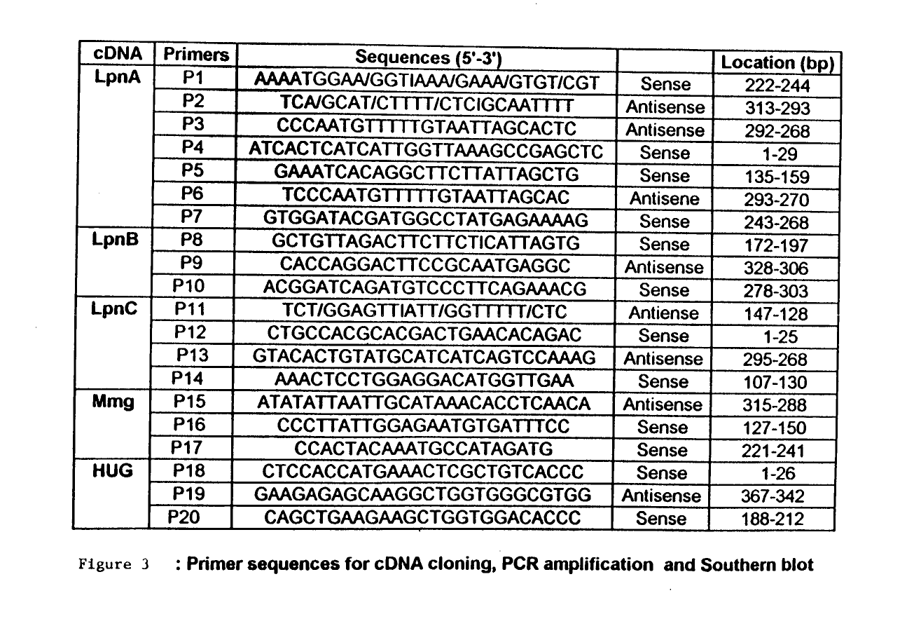

The amino acid sequence of lipophilins A and C are shown in FIG. 2. Lipophilin A contains 69 residues and a calculate pI of 9.47. Its calculated mass is in agreement with the measured mass of isolated lipophilin A, confirming that the peptide is not glycosylated or otherwise posttranslationally modified in tears. Lipophilin C contains 77 residues with a calculated pI of 4.94; its mass also matched that determined experimentally in tears.

Comparison of the N-terminal sequences of lipophilins A and C to the double sequence determined for residues 1-5 in the heterodimer shows that lipophilin A and lipophilin C monomers are associated head to head in forming the A-C dimers.

### EXAMPLE 3

### cDNA Cloning of Lipophilin A

Degenerate primers were used to amplify a human lachrymal gland cDNA library as follows. P1 (sense): 5′-AAAATGGA(A/G)GTIAA(A/G)AA(A/G)TG(T/C)GT-3′ and P2 (antisense): 5′-TC(A/G)CA(T/C)TTT(T/C)TCIGCAATTTT-3′. P1 corresponds to KMEVKKCV, and P2 was complementary to KIAEKCD. A TOPO TA kit (Invitrogen, Carlsbad, Calif.) was used to subclone the ≈95 bp PCR product, whose deduced sequence was identical to Lipophilin A.

A specific antisense primer P3 (5′-CCCAATGTTTTTGTAATTA GCA-CTC-3′) and the vector SK primer were used to amplify the human lachrymal gland library to obtain the 320 bp product containing 5′ -side untranslated region, signal sequence, and most of the mature lipophilin A cDNA sequence. The lachrymal gland library was amplified with 5′-side sense primer P4 (5′-ATCA-CTCATCATTGGTT AA AGCCGAG

CTC-3′), and vector T7 primer to complete the sequence. The cDNA obtained is shown in FIG. 4.

### EXAMPLE 4

### cDNA Cloning of Lipophilin C

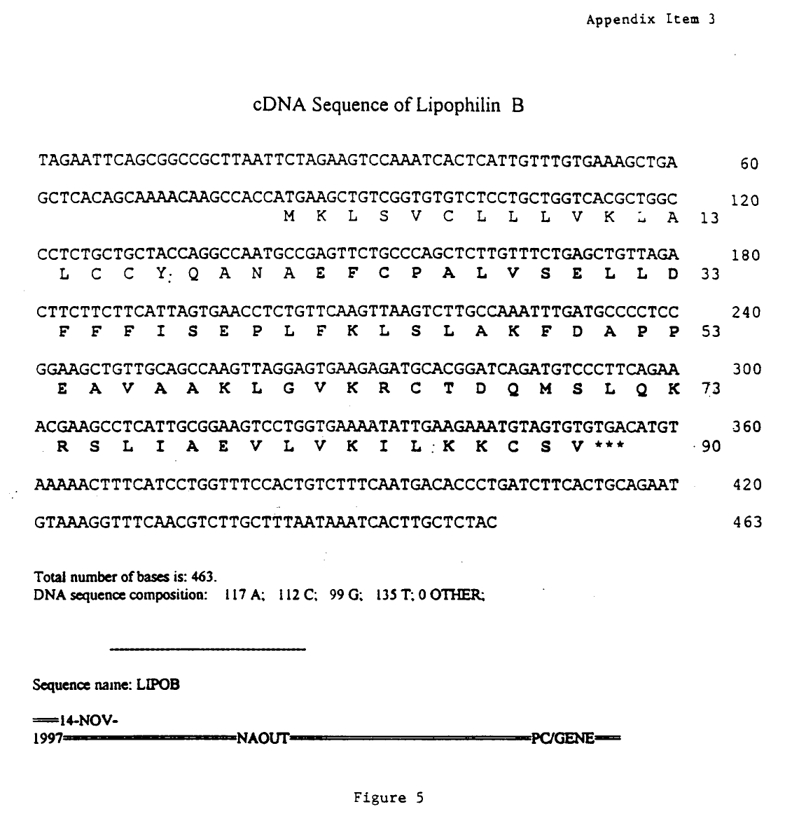

Lachrymal cDNA library DNA was amplified with vector SK primer and degenerate antisense primer P11(5′-TC(T/G)GAGTTIAT(T/G)GT TT T(T/C)TC) complementary to lipophilin C amino acids EKTINSD. The ≈145 bp. PCR product included some lipophilin C 5′ untranslated sequence, signal sequence and part of the mature peptide. To obtain the full sequence, 5 side sense primer P12 (CTGCCA-CGCA-CGA-CTGAA-CA-CAGA-C) and vector T7 primer were used to amplify lachrymal cDNA. The PCR fragments were subcloned and sequenced, providing the full 496-bp cDNA sequence shown in FIG. 5.

### EXAMPLE 5

### cDNA Cloning of Lipophilin B

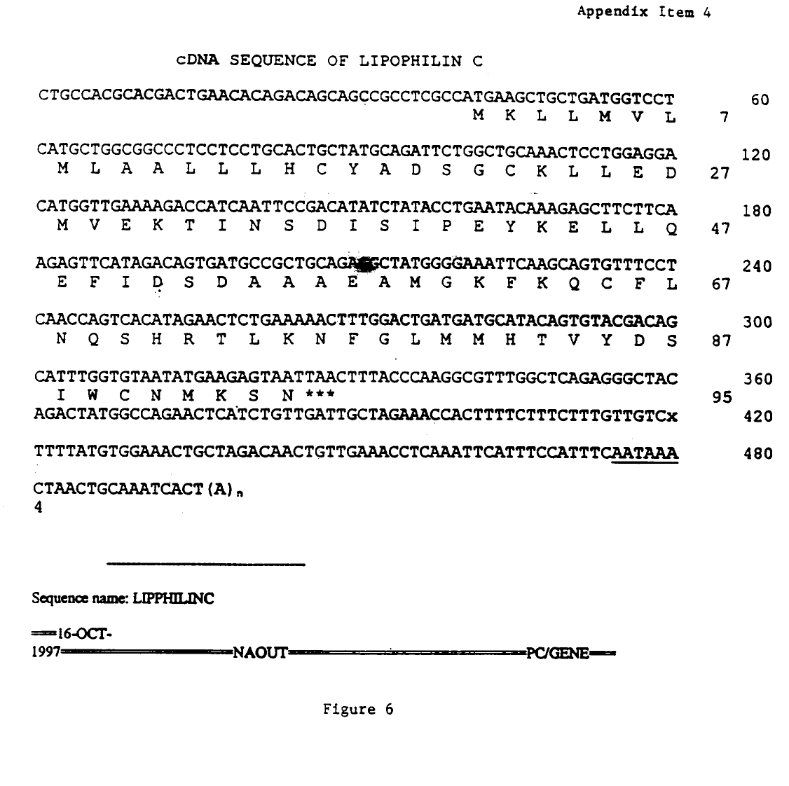

The 51 bp signal sequence of Lipophilin A was used to search the EST data base. An incomplete sequence (THC 210918) with high homology was used to design two primers: P8 (sense), 5′-GCTGTT AGA-CTTCTTCTICATTAGTG-3′), and P9 (anti sense), CA-CCAGGA-CTTCCG CAATGAGGC. Human uterus cDNA (Marathon-Ready™, Clontech, Palo Alto, Calif.) was amplified using 5′ and 3′ RA-CE PCR. P9 and adapter primers were used to get 5′-side cDNA, and P8 and adapter primers were used to get 3′-side cDNA, with a 150-bp sequence overlap between the two PCR products. The complete sequence of cDNA encoding lipophilin B is shown in FIG. 6.

### EXAMPLE 6

Comparison of Nucleotide and Amino Acid Sequences

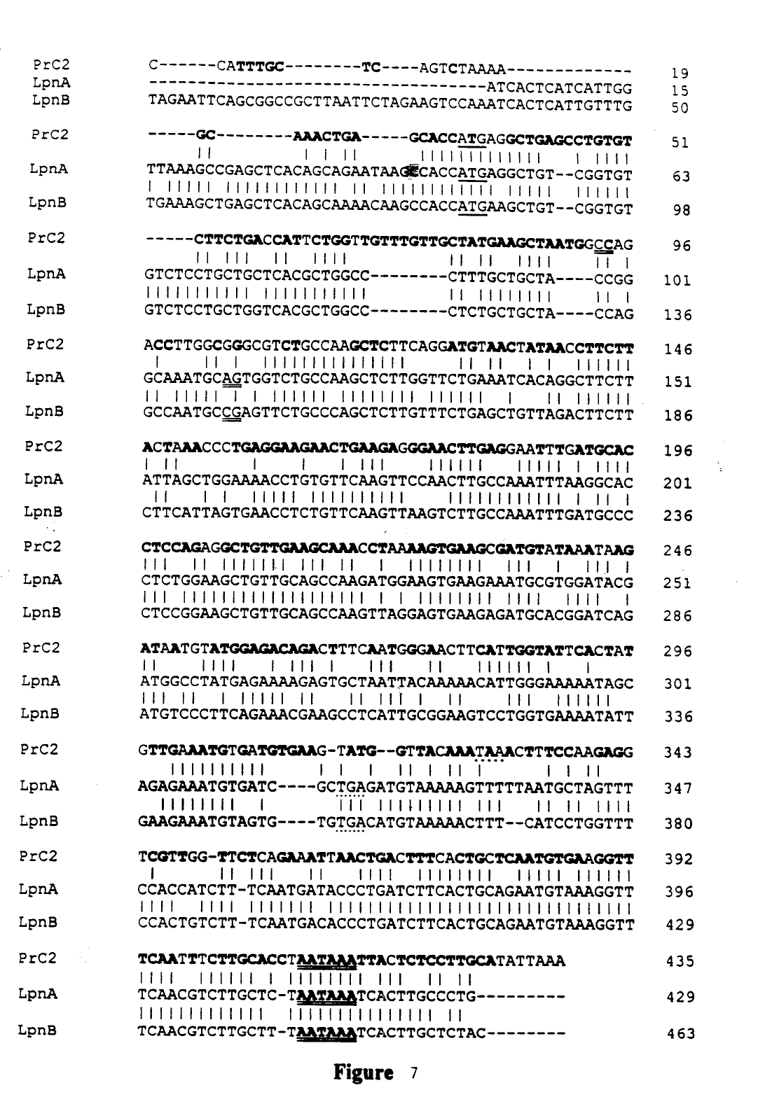

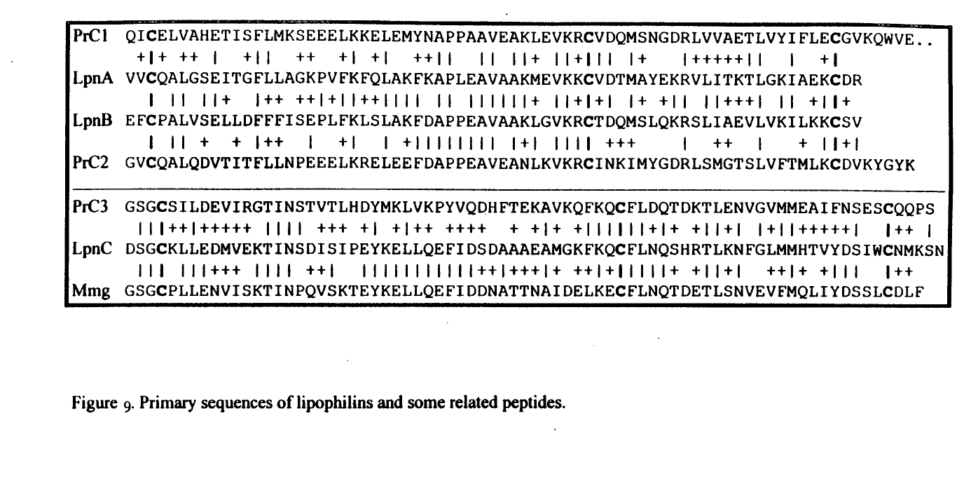

FIGS. 7, 8 and 9 show comparisons of the nucleotide sequences and amino acid sequences of the lipophilins A, B and C with proteins that are closely related to them.

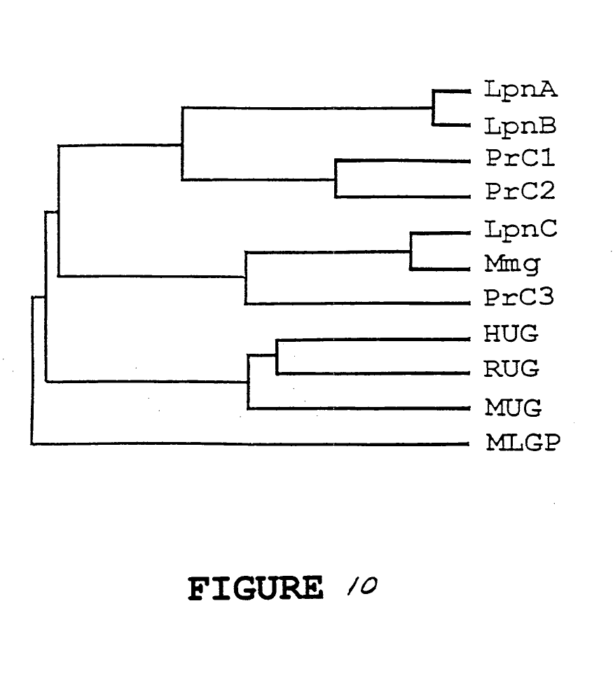

FIG. 10 is a dendrogram showing the level of relatedness of the lipophilins with the known prostatein peptides (PRC 1, PRC2 and PRC3) with mammaglobin (MMG) and with human, rat and murine uteroglobin. MLGP represents mouse lachrymal gland protein (GenBank AF008585).

### EXAMPLE 7

### Tissue Expression

Total human RNAs from human thymus, prostate, testis, salivary gland, skeletal muscle, small intestine, kidney, trachea, uterus, mammary gland, spleen, placenta, bone marrow, white blood cells, and ovary were purchased from Clontech (Palo Alto, Calif.). cDNA (20 μl) was synthesized from 1 μg of each total RNA with an Advantage™ RT-for-PCR kit (Clontech, Palo Alto). The PCR primers and the sequence locations are shown in Table 1. The human β-actin control amplimer set (Clontech) generated a 838 bp product. PCR was performed with an automated DNA thermal cycler for 35 cycles. Annealing temperatures from 55° C. to 70° C. were chosen according to the different primer sets. Reaction products (15 μl) were visualized after electrophoresis in 1.4% agarose gel containing 0.5 μg/ml ethidium bromide. Because several cDNA sequences studied here were highly homologous, different primer sets were designed, PCR analyses repeated for each cDNA for confirmation.

PCR products were electrophoresed in agarose, transferred onto nylon membranes (DuPont, Bost, Mass.), and hybridized them with digoxigenin labeled specific probes (P7 for Lipo A, P10 for Lipo B, P14 for Lipo C, P17 for human mammaglobin, and P20 for human uteroglobin). Labeling, washing and detecting used the nonradioactive DIG High Prime DNA Labeling and Detection Starter kit (Boehringer Mannheim, Indianapolis, Ind.), per the manufacturer's protocol. Light emission after alkaline phosphatase-mediated dephosphorylation of CSPD® was recorded on Hyperfilm™ MP (Amersham, Arlington Heights, Ill.) and analyzed by densitometry (Personal Densitometer SI, Molecular Dynamics, Sunnyvale, Calif.). To compare mRNA expression in different tissues, data were normalized to β-actin expressed by that tissue.

The results are shown in FIGS. 11A and 11B. As indicated, none of the lipophilins are appreciably produced in any cells which would interfere with assessment of metastases in the bloodstream. Lipophilin A was produced at high levels in the ovary, kidney, testis and thymus; lipophilin B was abundantly produced in ovary, breast, uterus, kidney, skeletal muscle, salivary gland, testis, prostate and thymus. Lipophilin C was highly produced in ovary, mammary gland, uterus, trachea, kidney, skeletal muscle, salivary gland, testis and prostate. As expected, mammaglobin is produced in particularly high levels in breast.

### EXAMPLE 8

### Expression of Lipophilins B and C by Prostate Cell Lines

A comparison of the levels of expression of lipophilins B and C in various prostate-derived cell lines with expression in normal prostate was determined by PCR. PCR was performed with an automated DNA thermal cycler for 35 cycles. Annealing temperatures 55° C. and 58° C. were chosen for lipophilin B and lipophilin C, respectively. A master reagent mix was used to ensure tube-to-tube consistency in PCR reactions in each experiment. Reaction products (5μl) were visualized after electrophoresis in 1.8% agarose gel containing 0.5μg/ml ethidium bromide. Polaroid pictures were taken from gels and analyzed by densitometry (ImageQuaNT Application, Molecular Dynamics, Sunnyvale, Calif.). To compare mRNA expression in different samples, data were expressed as percentages with normal prostate samples set at 100%.

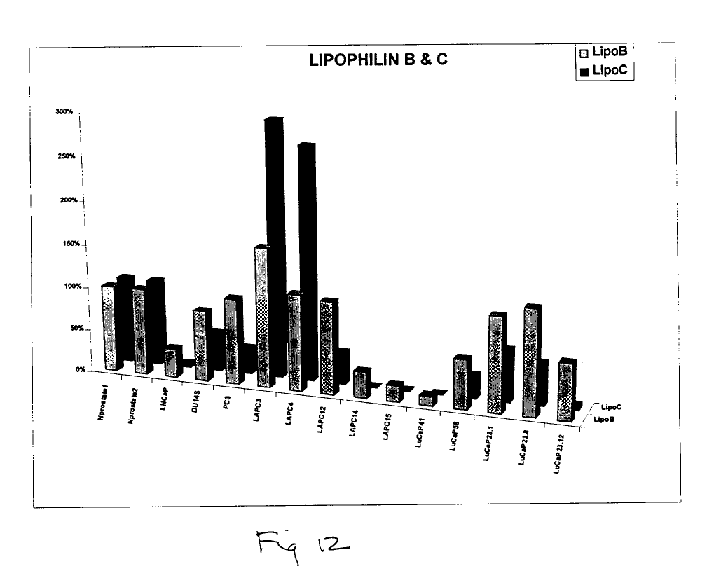

The results are shown in Table 1 and shown graphically in FIG. 12.

TABLE 1

| | Samples | LipB | LipC | |
|---|---|---|---|---|
| 1 | Nprostate1 | 100% | 100% | normal |
| 2 | Nprostate2 | 100% | 100% | prostate |
| 3 | LNCaP | 33% | 4% | prostate |
| 4 | DU145 | 81% | 45% | cancer cell |
| 5 | PC3 | 99% | 30% | lines |
| 6 | LAPC3 | 161% | 295% | prostate |
| 7 | LAPC4 | 111% | 269% | cancer |
| 8 | LAPC12 | 106% | 37% | xenografts |
| 9 | LAPC14 | 30% | 0% | |
| 10 | LAPC15 | 19% | 0% | |
| 11 | LuCaP41 | 11% | 0% | |
| 12 | LuCaP58 | 57% | 26% | |
| 13 | LuCaP23.1 | 108% | 59% | sublines of |
| 14 | LuCaP23.8 | 120% | 49% | xenografts |
| 15 | LuCaP23.12 | 65% | 5% | |

As shown by numerical data in Table 1 and graphed in FIG. 12, all of 13 prostate tumor samples tested showed an abnormal level of expression of lipophilin B or C. Decreased lipophilin C expression at less then 50% of normal was shown in 76.9% of the samples; 4 of the 10 samples with diminished expression of lipophilin C also had greatly diminished expression of lipophilin B. In 2/13 (15.4%) of the samples there was a markedly increased expression of lipophilin C and one of these also showed increased expression of lipophilin B.

### EXAMPLE 9

### Chromosomal Localization

A of Lipophilin A was used to screen a human placenta cosmid library (Clontech,Palo Alto, Calif.). Seven positive clones were chosen. Southern blots performed with specific oligonucleotide probes (P5, lipophilin A; P8, lipophilin B) identified 3 as lipophilin A clones and 4 as lipophilin B. The entire genomic clone of Lipophilin B was used to perform fluorescence in situ hybridization (See DNA Biotech, Toronto, Canada BrdU treated, phytohemagglutin-stimulated normal human lymphocytes, using procedures described by Heng and Tsui, Chromosoma (1993) 102:325-332.

The cDNA sequences of lipophilins were deposited with EMBL Nucleotide Sequence Database, with the following accession numbers: Lpn A, AJ224171; Lpn B, AJ224172; Lpn C, AJ224173. The deposit is in confidence pending manuscript acceptance.

1. A peptide in purified and isolated form or the n-terminal acylated and/or C-terminal amidated and fusion protein forms thereof comprising the amino acid sequence of human lipophilin a, b or c or an allelic variant thereof or a contiguous fragment thereof, including the n-terminal acylated and/or c-terminal amidated or fusion protein forms of said fragment wherein said fragment comprises at least one immunogenic epitope of said peptide:

2. The peptide or fragment of claim 1 which is coupled to an immunogenicity enhancing component.

3. The peptide or fragment of claim 1 which is coupled to a solid support.

4. A nucleic acid comprising a nucleotide sequence that encodes the peptide or fragment of claim 1 or the complement thereof.

5. A recombinant expression system which expression system comprises a nucleic acid containing the encoding nucleotide sequence of claim 4 operably linked to control sequences for effecting its expression.

6. Recombinant host cells which comprise the expression system of claim 5.

7. A method to prepare a peptide comprising the amino acid sequence of human lipophilin A, B or C or an allelic variant thereof or a fragment thereof comprising at least one immunogenic epitope which method comprises culturing the cells of claim 6 under conditions wherein said peptide or fragment is produced and optionally recovering said peptide or fragment.

8. An oligonucleotide probe which comprises the nucleotide sequence of a portion of the nucleotide sequence of a cDNA corresponding to human lipophilin A, B or C or the complement thereof of sufficient length to hybridize, under conditions of sufficient stringency to eliminate false positives, to mRNA corresponding to lipophilin A, B or C or to a reverse transcript thereof.

9. A method to detect cells in a body fluid or biopsied tissue of a subject that express the gene for lipophilin A, B or C which method comprises
obtaining mRNA or a reverse transcript thereof from said body fluid or tissue;
contacting said mRNA or reverse transcript with the probe of claim 8 under conditions wherein said mRNA or reverse transcript hybridizes to said probe; and
detecting the presence or absence of a complex;
wherein the presence of the complex indicates the presence of cells expressing the gene for lipophilin A, B or C.

10. The method of claim 9 wherein the body fluid is blood, plasma or serum and the biopsied tissue is bone marrow or lymph nodes.

11. The method of claim 9 wherein said probe is derived from lipophilin B or C.

12. A kit for the detection of tumor metastases in humans which kit comprises a container which contains the probe of claim 8 along with reagents for detecting hybridization between said probe and mRNA or its reverse transcript if present in said body fluid or tissue.

13. A method to detect metastases in blood, plasma or serum which method comprises
contacting a sample of said blood, plasma or serum or suitable fraction thereof with the probe of claim 8 and
detecting hybridization between said probe and mRNA or reverse transcript corresponding to lipophilins A, B or C,
whereby the presence of said hybrid indicates the presence of metastases.

14. A pair of oligonucleotide primers for amplification of a characteristic portion of a nucleotide sequence contained in mRNA corresponding to human lipophilin A, B or C or the complement thereof.

15. A method to detect the presence of cells expressing the gene for lipophilin A, B or C in a body fluid or biopsied tissue of a subject which method comprises
obtaining mRNA or a reverse transcript thereof corresponding to said lipophilin A, B or C from said body fluid or tissue;
conducting a reaction to amplify a characteristic portion of said mRNA or reverse transcript in the presence of the primers of claim 14; and
detecting the presence or absence of an amplification product;
wherein the presence of an amplification product indicates the presence of cells expressing said lipophilin A, B or C.

16. The method of claim 15 wherein said body fluid is blood, plasma or serum and the biopsied tissue is bone marrow or lymph nodes.

17. The method of claim 15 wherein said primers correspond to lipophilin B or C.

18. A kit for the detection of tumor metastasis in humans which kit comprises a container which contains the primers of claim 14 along with means for detecting an amplification product of a characteristic portion of mRNA or its reverse transcript corresponding to human lipophilin A, B or C.

19. A method to detect metastases in blood, plasma or serum which method comprises
contacting a sample of said blood, plasma or serum or suitable fraction thereof with the primers of claim 14 under conditions wherein a nucleotide sequence appropriate to said primers is amplified and
detecting an amplification product of a characteristic portion of mRNA or its reverse transcript corresponding to human lipophilin A, B or C whereby the presence of an amplification product indicates that metastases are present.

20. Antibodies or immunoreactive fragments thereof which are monospecific for the peptide or fragment of claim 1.

21. The antibodies or fragments of claim 20 wherein said antibodies are monoclonal antibodies.

22. A population of cells that secrete the antibodies of claim 21.

23. The population of claim 22 wherein said cells are hybridomas.

24. A method to detect cells in a body fluid or biopsied tissue of a subject that express lipophilin A, B or C which process comprises
obtaining sample of the peptide from said body fluid or tissue;
contacting said peptides with the antibody or fragments of claim 20 under conditions wherein a complex is formed between said antibodies or fragments and peptides immunoreactive therewith;
detecting the presence or absence of said complex; wherein the presence of the complex indicates the presence of cells expressing the gene for lipophilin A, B or C.

25. The method of claim 24 wherein the body fluid is blood, plasma or serum and the biopsied tissue is bone marrow or lymph nodes.

26. The method of claim 24 wherein the antibodies or fragments are immunoreactive with lipophilin B or C.

27. A kit for the detection for tumor metastases in humans which kit comprises a container which contains the antibodies or fragments of claim 20 along with reagents for detecting a reaction between said antibodies or fragments and peptides immunoreactive therewith in a body fluid or biopsied tissue.

28. A method to detect metastases in blood, plasma or serum which method comprises contacting said blood, plasma or serum or suitable fraction thereof with the antibodies or fragments of claim 20 and detecting formation of a complex between said antibodies or fragments and peptides immunoreactive therewith in said sample whereby the formation of said complex indicates the presence of metastases.
